(19) United States
(12) Patent Application Publication
(10) Pub. No.: US 2013/0115253 A1
(43) Pub. Date: May 9, 2013

(54) Sustained Release Suspension Preparation For Dextromethorphan

(71) Applicants: Mahendra R. Patel, Milltown, NJ (US); Amol S. Matharu, Edison, NJ (US)

(72) Inventors: Mahendra R. Patel, Milltown, NJ (US); Amol S. Matharu, Edison, NJ (US)

(21) Appl. No.: 13/671,007

(22) Filed: Nov. 7, 2012

Related U.S. Application Data
(60) Provisional application No. 61/556,559, filed on Nov. 7, 2011.

(51) Int. Cl. A61K 47/48

(52) U.S. Cl. CPC A61K 47/48776
USPC 424/400; 424/78.14

(57) ABSTRACT

A stabilized pharmaceutical composition comprises a drug-resin complex wherein the resin has been treated with an alkaline material prior to the formation of the drug-resin complex. The drug-resin complex may further be impregnated with an alkalizing agent, L-methionine, an antioxidant agent, or a combination thereof, or be coated with a diffusion barrier. A method of preparation of the pharmaceutical composition is provided.

### FIELD OF THE INVENTION

The present invention relates generally to a pharmaceutical composition. More specifically, the invention relates to a sustained release drug-resin suspension that possesses an improved stability and a method of making the composition.

### BACKGROUND OF THE INVENTION

Pharmaceutical dosage forms are essential in safely administering active agents such as drugs. Appropriate dosage forms can optimize bioavailability, provide a desirable drug dissolution profile, improve dosage consistency, and improve patient compliance (e.g., by reducing dosing frequency). Commonly used pharmaceutical dosage forms include solid, liquid, or semi-solid dosage forms. For prolonged and sustained drug delivery, a resin-drug complex in a suspension or capsule is often used.

Resins are water-insoluble polymers in the forms of very small particles and beads. The most commonly used resin for a drug-resin complex is ion exchange resin, which contains salt-forming groups in repeating positions on the resin chain. Generally, a cationic exchange resin is used to prepare a drug-resin complex with a basic drug, and an anionic exchange resin is used to prepare a drug-resin complex for an acidic drug. Additionally, an amphoteric ion exchange resin may be used.

A resin-drug complex is prepared by mixing a resin with a drug solution, either by repeated exposure of the resin to the drug in a chromatographic column or by prolonged contact of the resin with the drug in a container. Drug molecules attached to the resin can later be exchanged by appropriately charged ions in contact with the ion-exchange groups, and the released drug molecules diffuse out of the resin. This process is known as drug release.

Drug release from a resin-drug complex depends on the ionic environment, such as pH or electrolyte concentration, within the gastrointestinal tract, as well as properties of the resin. In general, a drug-resin complex often dissolves more slowly than an ordinary drug formulation. All these properties make a drug-resin complex useful in changing drug dissolution profiles. As such, a drug-resin complex is frequently used in time-release formulations.

The drug release rate can be further modified by coating the drug-resin complex by a microencapsulation process. Different coating materials alter the drug release rate differently. For instance, a drug-resin complex can be coated with an enteric coating polymer, which protects the drug composition contained therein against the acid environment of the stomach and then provides release of the drug in the small intestine. Coated and uncoated drug-resin complexes can be mixed and filled into capsules with excipient or suspended in a palatable, flavored suspension. By controlling the ratio of the coated and uncoated drug-resin complexes, a desired drug release profile may be obtained.

Besides providing sustained release, a drug-resin complex has several other advantages over pure drugs in ordinary formulations. Many drugs are bitter and/or smell bad, such as Bromhexin and Quinine. One advantage of formulating a drug into a drug-resin complex is that the bitterness or the bad smell of a drug may be masked.

However, many drugs, which are otherwise stable, have been found to be unstable when bound to a resin. The resulting degradant from the original drug can be an oxidized form or hydrolytic form of the drug. The degradation may occur when the drug-resin is dry or wet.

Several methods are known to stabilize drug compositions. U.S. Pat. No. 5,980,882 (Eichman) describes the use of chelating agents such as EDTA (ethylene diamine tetra-acetic acid) to stabilize a pharmaceutical composition composed of a drug-resin complex. It is known that the oxidation and hydrolytic reactions of many drugs are catalyzed by metal ions. Chelating agents, as scavengers for trace amounts of metal ions, are useful in preventing drug degradation. Additionally, the anti-microbial activity of EDTA also contributes to the stability of a drug-resin ophthalmic composition according to U.S. Pat. No. 5,182,102 (DeSantis, Jr. et al.) and U.S. Pat. No. 5,540,918 (Castillo et al.).

U.S. Pat. No. 4,973,607 (Stahlbush et al.) describes the use of an antioxidant to improve the chemical stability of a cationic exchange resin.

The use of a preservative to stabilize drugs is known. U.S. Pat. No. 5,368,852 (Umemoto et al.) teaches the use of sodium benzoate and the use of an ethylcellulose coating on a drug-resin complex to reduce bacterial activity. U.S. Pat. No. 4,448,774 (Clemente et al.) teaches the use of sodium benzoate as a preservative and the use of EDTA as a chelating agent to stabilize an aqueous pharmaceutical formulation.

Coating the beads formed by drug-resin complexes is a necessary step of formulation to control release of the drug for a prolonged time. Ethylcellulose has been taught as a barrier-forming material of choice. U.S. Pat. No. 4,221,778 (Raghunathan) and U.S. Pat. No. 4,847,077 (Raghunathan), and Raghunathan et al. in J. Pharm. Sci., Vol 70, pp 379-384, April 1981, describe the coating of drug-ion exchange resin complexes with a water-permeable diffusion barrier such as ethycellulose.

Formulation principle of Delsym® has been well discussed in Borodkin, S. Book chapter: Ion-exchange resin delivery system, in “Polymers for Controlled Drug Delivery”, Tarcha, P J, Ed., CRC Press, Boca Raton, 1990. The effect of coating levels on Delsym® in-vivo performance has shown to be dependent on coating levels.

As a result of the degradation, the shelf life of a pharmaceutical composition comprising a drug-resin complex is generally not long. This also means that patients and prescribers are unable to judge the dosage strength with accuracy.

Thus, there remains an unmet need for a pharmaceutical composition comprising a drug-resin complex that provides improved drug stability over a long period of time.

### SUMMARY OF THE INVENTION

The invention provides a stabilized pharmaceutical composition comprising a drug-resin complex, wherein the resin has been treated with an inorganic alkaline material, preferably sodium hydroxide, prior to the formation of the drug-resin complex. The drug-resin complex may further be impregnated with an alkalizing agent, preferably magnesium oxide, be impregnated with L-methionine, an antioxidant agent, or a combination thereof, or be coated with a diffusion barrier coating prior to final formulation to improve the drug stability or modify the drug dissolution profile. It is discovered that the addition of phosphoric acid in the formulation improves the drug stability, while the use of propylene glycol decrease the drug stability of the composition.

The invention also provides a method of preparing a stabilized pharmaceutical composition comprising the steps of: (1) treating a resin with an inorganic alkaline material; (2) combining a drug and the treated resin to form a drug-resin complex; (3) formulating the complex from the previous step to form a pharmaceutical composition. Prior to the formulating of step (3), the drug-resin complex may be impregnated with an alkalizing agent, be impregnated with L-methionine, an antioxidant agent, or a combination thereof, or be coated with a diffusion barrier coating to further improve the drug stability or modify the drug dissolution profile.

### BRIEF DESCRIPTION OF THE DRAWINGS

FIG. 1 is a graph illustrating the comparative dissolution profiles of dextromethorphan from the extended release formulation in accordance with the present invention and from the commercial Delsym cough suppressant.

### DETAILED DESCRIPTION OF THE INVENTION

The present invention is based on the finding that the stability of a drug-resin complex improves significantly when the resin is treated with an inorganic alkaline material prior to use. The stability of the drug-resin complex may further be improved by impregnating the drug-resin complex prior to final formulation. The drug-resin complex may further be coated with a diffusion barrier to obtain a desirable drug dissolution profile.

In one aspect, the present invention provides a pharmaceutical composition with an improved stability that comprises a drug-resin complex, wherein the resin used therein has been treated with an inorganic alkaline material and wherein the drug-resin complex has been impregnated with an alkalizing agent and optionally a solvating agent.

In another aspect, the present invention provides a pharmaceutical composition with an improved stability that comprises a drug-resin complex, wherein the resin used therein has been treated with an inorganic alkaline material and wherein the drug-resin complex has been impregnated with L-methionine, an antioxidant agent, or a combination thereof. The drug stability may further be improved by adding phosphoric acid and by avoiding use of propylene glycol in the formulation.

In yet another aspect, the present invention provides a pharmaceutical composition with an improved stability that comprises a drug-resin complex, wherein the resin used therein has been treated with an inorganic alkaline material and wherein the drug-resin complex has been coated with a diffusion barrier to modify the drug dissolution profile.

In a further aspect, the invention provides a method of preparing a pharmaceutical composition with improved drug stability. The method comprises the steps of: (1) treating a resin with an inorganic alkaline material; (2) combining a drug and the treated resin to form a drug-resin complex; (3) impregnating or coating the drug-resin complex with suitable reagents to either improve the stability or modify the dissolution profile of the drug; and (4) formulating the complex from the previous step to form a pharmaceutical composition.

In accordance with the present invention, any non-toxic resin may be suitable for use as long as a drug can be sufficiently bound or adsorbed into the resin. A preferred resin is ion exchange resin. Ion-exchange resins suitable for use in the present invention are water-insoluble and preferably comprise pharmacologically inert organic and/or inorganic matrix containing functional groups that are ionic or capable of being ionized under the appropriate conditions of pH. The organic matrix may be synthetic (e.g., polymers or copolymers of acrylic acid, methacrylic acid, sulfonated styrene, sulfonated divinylbenzene), or partially synthetic (e.g. modified cellulose and dextrans). The inorganic matrix preferably comprises silica gel modified by the addition of ionic groups. Covalently bound ionic resins may also be used. The covalently bound ionic groups may be strongly acidic (e.g., sulfonic acid, phosphoric acid), weakly acidic (e.g., carboxylic acid), strongly basic (e.g., primary amine), weakly basic (e.g. quaternary ammonium), or a combination of acidic and basic groups. Such ion-exchangers are described by Borodkin, S. Book chapter: Ion-exchange resin delivery system, in “Polymers for Controlled Drug Delivery”, Tarcha, P J, Ed., CRC Press, Boca Raton, 1990.

An ion exchange resin known to be useful in the present invention is divinylbenzene sulfonic acid cationic exchange resin, in either sodium salt or potassium salt form. In one embodiment, Purolite C 100 E MR/4395 is used which has a particle size less than 150 micron. Other commercially available equivalent resins that can be used are Amberlite IRP-69 and Dow XYS-40010.00. Both are sulfonated polymers composed of polystyrene cross-linked with about 8% of divinylbenzene, with an ion-exchange capacity of about 4.5 to 5.5 meq/g of dry resin (H⁺ form). Their essential difference is in physical form. Amberlite IRP-69 consists of irregularly shaped particles with a size range of about 5 microns to about 149 microns produced by milling the parent large size spheres of Amberlite IRP-120. The Dow XYS-40010.00 product consists of spherical particles with a size range of 45 microns to 150 microns.

All drugs which exist in an ionic form may be used to bind with ion exchange resins in the present invention. Such drugs include, but are not limited to, many families of drugs such as antibacterials, antivirals, antifungals, anti-parasitics, tumoricidals, anti-metabolites, polypeptides, immunoglobulins, immunomodulators, vasodilators, anti-inflammatories, antiglaucomics, mydriatic compounds, antidepressants, antispasmodics, antiulceratives, anxiolytics, calcium channel blockers, dopamine receptor agonists and antagonists, narcotic antagonists, protease inhibitors, respiratory stimulants, retroviral protease inhibitors, reverse transcriptase inhibitors.

The drugs that in particular are benefited from the present invention are those prone to degradation after complexation, for example, due to oxidation or hydrolysis. In some embodiments, the drug is selected from a group consisting of dextromethorphan, codeine, morphine, hydrcodone, pseudoephedrine, phenylpropanolamine and the salts thereof. In a preferred embodiment, the drug is dextromethorphan.

In accordance with the present invention, the resin, before being complexed with the drug, is treated with an inorganic alkaline material in water. It is observed that a drug-resin complex which is made of the treated resin exhibits significantly improved stability compared to a drug-resin complex using an untreated resin.

The treatment can be conducted by soaking, preferably with stirring, the resin in an aqueous solution of an inorganic alkaline material for an extended time. An elevated temperature may be used to increase the effectiveness of the treatment. The concentration of the inorganic alkaline material may vary. In one embodiment, the inorganic alkaline material is 2N sodium hydroxide. The time required for the treatment also vary, depending on the amount of resins to be pretreated, the temperature, the time, the type of the inorganic alkaline material and its concentration.

The treatment can also be conducted in a chromatographic column by repeatedly running an aqueous solution of an inorganic alkaline material through the chromatographic column containing the resin to be treated.

The alkalizing material are, preferably, pharmaceutically acceptable inorganic salts of alkaline metals and alkaline earth metals such as lithium salts, potassium salts, sodium salts in form of oxide, carbonate, bicarbonate, and the like. Most preferably alkalizing agent for the resin is sodium hydroxide.

All water used for the invention is preferably distilled or purified water, free of minerals, ions, and ion exchange components may be used for the invention. In a preferred embodiment, deionized (DI) water is used.

After the alkaline pretreatment, the treated resin is collected by filtration, optionally, the resin is washed with water numerous times. The resin is then dried at about 50° C. until the water content is less than 8%, as measured by the well known Karl-Fischer method. The dried, treated resin can then be reacted with a drug to form a drug-resin complex using standard techniques.

In one embodiment, a drug-resin complex is formed by adding an aqueous solution of a drug to a container containing the treated resin and stirring for sufficient time. The resulting drug-resin complex suspension is filtered, and optionally is washed with water numerous times, to yield the drug-resin complex. The drug-resin complex is then dried until the water content was below 8%, as measured by the Karl-Fischer method.

The amounts of drug and resin necessary to form an effective drug resin complex vary greatly. Among the factors to be considered in determining the ratio of drug to resin are the particular drug itself, the resin, the reaction conditions, and the final dosage form. The resin preferably has a high loading capacity for the drug in question. A small loading capacity may make the resulting dosage form overly bulky or expensive to produce. Actual loading of the drug on the resin particles can range from about 1% to 90%by weight but preferably 5% to 30% by weight of the resin.

The stability of the drug-resin complex is also improved by impregnating the drug-resin complex with an alkalizing agent.

Suitable alkaline agents for the present invention can be organic or in-organic agents. Inorganic alkaline agents include, but not limited to, carbonate, alkaline oxide, and hydroxide. Preferred inorganic alkaline agents include MgO, Mg(OH)₂, CaCO₃, Ca(OH)₂, MgCO₃. Even more preferably, it is magnesium oxide (MgO). Organic alkaline agents include, but not limited to, pseudoephedrine and phenylpropanolamine.

The alkalizing agent typically is present in an amount of about 5% to about 30% by weight of the resin. Preferably, it is about 10% by weight of the resin.

Additionally, the drug-resin complex may be treated with a solvating agent to aid the impregnation of the alkalizing agent. This step can be performed simultaneously with or before the impregnation of magnesium oxide. Preferable solvating agents include polyol, such as polyethylene glycol, glycerol, propylene glycol. Polyethylene glycol (PEG), in particular, PEG 3350, is preferred. The solvating agent may be in an amount of about 5% to about 35% by weight of the resin. Preferably, it is about 15% by weight of the resin.

It has been observed that the alkalized drug-resin complex, i.e., the complex which has been impregnated with PEG and MgO, exhibits significantly improved stability, compared to the drug-resin complex without such impregnation.

The drug-resin complex made from a resin pre-treated with an inorganic alkaline material can further be impregnated with L-methionine. For better result, a solvating agent is added in this step. It is observed that the impregnation with L-methionine and a solvating agent improves the drug stability of the pharmaceutical composition. In one embodiment, the impregnation is conducted by adding a few drops of water to moisten the mixture of L-methionine, PEG and the alkalized drug-resin complex, thoroughly mixing the ingredients, and then drying. For better result, the L-methionine and PEG are grounded to fine powders prior to use. The L-methionine may be in an amount of about 5% to about 30%, preferably, about 10% by weight of the resin. The PEG amount used is about 5% to about 35%, preferably, about 15% by weight of the resin.

Other ingredients, such as an antioxidant, can also be impregnated simultaneously with L-methionine. Antioxidant is known to improve the chemical stability of resins. Possible antioxidant agents include, but not limited to, butylated hydroxyanisole (BHA) and butylated hydroxytoluene (BHT), with BHT being the preferred one. The antioxidant is generally used in an amount of about 0.05% to about 0.5%, preferably, about 0.2% by weight of the resin. It should be noted that, antioxidant can also be added to the final formulation, rather than being impregnated onto the resin, to improve the stability of the composition.

The alkalized drug-resin complex of the present invention can be formulated into any pharmaceutical dosage forms for oral, topical, rectal, vaginal, nasal, or ophthalmic administration. Preferred dosage forms include syrups and suspensions. Commonly known ingredients and procedures to formulate a drug-resin pharmaceutical composition are within the purview of a person skilled in the art. Various methods as described in U.S. Pat. Nos. 4,221,778, 4,762,709, 4,788,055, 4,959,219, 4,996,047, 5,071,646, and 5,186,930 are incorporated herein in their entirety by reference herein, can be used to formulate the composition.

The stability of drug-resin pharmaceutical suspensions is tested. Of the commonly known ingredients used in formulating a suspension, propylene glycol is found to destabilize the suspension, and phosphoric acid is found to stabilize the suspension.

The present invention further provides a pharmaceutical composition having not only a stabilized drug-resin complex but also a desirable dissolution profile. This is achieved by coating the alkalized drug-resin complex with a film forming polymer prior to the final formulation. The film forming polymer is called a diffusion barrier because it can slow the rate of drug dissolution. Possible coating materials include, but not limited to, hydoxypropyl cellulose (HPC), ethylcellulose, methylcellulose, polyethylene glycol, mannitol, lactose and others, with HPC being the preferred coating material. Additionally, a functional coating may be used to further control the dissolution, for instance, to sustain or delay the release of the drug from the drug-resin complex. Varying the amount of coating or combining coated and uncoated complexes in the same formulation can be used to adjust the dissolution profile as desired.

Conventional coating procedures such as those described in U.S. Pat. No. 4,221,778, whose entire contents are incorporated by reference herein, can be used to coat the particles. In one embodiment, the coating is carried out in a fluid bed coating apparatus equipped with a Wurster Column. Samples are collected at three intervals in order to assess the coating weight gain influenced release. Following coating, the complex is well mixed with colloidal silicon dioxide at 1%, followed by curing in a forced draft oven for 48 hours at 40 ° C.

The coated drug-resin complex is formulated into a desirable pharmaceutical dosage form. The pharmaceutical composition prepared in accordance with the present invention is able to maintain a sustained release profile that is comparable to a brand name product.

The present invention is further illustrated by the following Examples.

### EXAMPLE 1

### Preparation of A Drug-Resin Complex

1A. Alkali Treatment of a Resin

To 100 g of crosslinked polystyrene divinylbenzene resin (Purolite C 100 E MR/4395), 250 mL of 2N sodium hydroxide was added and the resulting mixture was stirred at 50° C. for 7 hours. The mixture was then filtered to yield the treated resin. The treated resin was dried at 50° C. until the water content was less than 8%, as measured by the Karl-Fischer method.

1 B. Drug Loading of the Alkali Treated Resin

To 100 mg of the treated resin obtained in Example 1A, 40 mg of dextromethorphan hydrobromide monohydrate was added as a solution in 100 mL of water to form a treated drug-resin complex suspension in water. The resulting mixture was filtered and the resulting treated drug-resin complex was dried to water content below 8%, as measured by the Karl-Fischer method.

It should be noted that the above scale is exemplary. In practice, the preparation can be scaled up or down depending on the final quantity of the treated drug-resin complex.

1C. Drug Loading of an Untreated Resin

To 40 mg of crosslinked polystyrene divinylbenzene resin (Purolite C 100 E MR/4395), 100 mg of dextromethorphan hydrobromide monohydrate was added as a solution in 100 mL of water to form an untreated drug-resin complex suspension in water. The resulting mixture was filtered and the resulting untreated drug-resin complex was dried to water content below 8%, as measured by the Karl-Fischer method.

It should be noticed that the above scale is exemplary. In practice, the preparation can be scaled up or down depending on the final quantity of the untreated drug-resin complex.

### EXAMPLE 2

### Stability Comparison of the Treated and Untreated Drug-Resin Complexes

Stabilities of the dextromethorphan polystirex drug-resin suspension samples prepared from the untreated resin (Example 1C) and from the alkali treated resin (Example 1B) were compared. The effect of impregnating with a soaking agent, polyethylene glycol (PEG), and magnesium oxide, to both treated and untreated drug-resin complexes, was also evaluated.

Stability testing after storing the suspension at 50° C. for 1 month was performed to establish stability. The stability test measures the % of (−)-3-methyl-10-oxy-methylmorphinan (“the dextromethorphan ketone”), which is a degradation product of dextromethorphan, via HPLC. The test results are shown in Table 1.

TABLE 1
Dextromethorphan-polistirex degradation study

| | % ketone degradation |
|---|---|
| Sample description | product at |
| (30 mg dextromethorphan/5 mL water) | 50° C./1 month |
| Example 1C (Untreated Drug-resin complex) in | 42.23 |
| 5 mL water | |
| Example 1B (Treated Drug-resin complex) in | 16.29 |
| 5 mL water | |
| Example 1C (Untreated Drug-resin complex) | 44.0 |
| with 10% PEG in 5 mL water | |
| Example 1B (Treated Drug-resin complex with | 20.85 |
| 10% PEG in 5 mL water | |
| Example 1C (Untreated Drug-resin complex) | 3.1 |
| with 10% PEG and 10% MgO, in 5 mL water | |
| Example 1B (Treated Drug-resin complex) with | 0.99 |
| 10% PEG and 10% MgO, in 5 mL water | |

The data in Table 1 show that the drug-resin complexes made from a treated resin is consistently more stable than the drug-resin complexes made from an untreated resin. The results also show that the presence of PEG had minimal effect in improving stability. However, addition of MgO to the drug-resin complex substantially improves stability.

### EXAMPLE 3

### Impregnating the Drug-Resin Complex

3A. Impregnating with Butylated Hydroxytoluene (BHT) (0.2 mg), L-methionine and PEG 3350

The alkaline treated dextromethorphan-polystirex complex from Example 1B was impregnated with BHT, PEG 3350 and L-Methionine according to the formulation of Table 2. It would be obvious to a person skilled in the art that Table 2 illustrates the relative proportion of each ingredient to be use for making a 5 mL product. In practice, the amount of each ingredient will be proportionally scaled up in making a large amount of the final product.

The impregnation procedure was as follows: in three separate mortars and pestles, ground BHT, PEG 3350, and L-methionine to make fine powders of each. Weighed the appropriate amount of BHT, PEG 3350, and L-methionine, mixed them thoroughly in a container. To the container, added the treated dextromethorphan - polystirex complex from Example 1B. Added a few drops of DI Water to moisten the mixture and stirred well. Dried the mixture at room temperature.

TABLE 2
The materials for the impregnation, Example 3A

| | Quantity in | |
|---|---|---|
| Name of Materials: | 5 mL dosage | |
| BHT | 0.2 | mg |
| PEG 3350 | 14 | mg |
| L-Methionine | 10 | mg |
| The drug-resin complex | 97 | mg |
| from Example 1B | | |

3B. Impregnating with L-Methionine and PEG 3350

The alkaline treated dextromethorphan-polystirex complex from Example 1B was impregnated with L-Methionine and PEG according to the formulation of Table 3. The procedure used was similar to Example 3A.

TABLE 3
The materials for the impregnation, Example 3B

| | Quantity in |
|---|---|
| Name of Materials: | 5 mL dosage |
| PEG 3350 | 14 mg |
| L-Methionine | 10 mg |
| The drug-resin complex | 97 mg |
| from Example 1B | |

3C. Impregnating with BHT (0.4 mg), L-Methionine and PEG 3350

The alkaline treated dextromethorphan-polystirex complex from Example 1B was impregnated with BHT, L-Methionine and PEG according to the formulation of Table 4. The procedure used was similar to Example 3A.

TABLE 4
The materials for the impregnation, Example 3C

| | Quantity in | |
|---|---|---|
| Name of Materials: | 5 mL dosage | |
| BHT | 0.4 | mg |
| PEG 3350 | 14 | mg |
| L-Methionine | 10 | mg |
| The drug-resin complex | 97 | mg |
| from Example 1B | | |

3D. Impregnating with BHT (0.2 mg) and PEG 3350

The alkaline treated dextromethorphan-polystirex complex from Example 1B was impregnated with BHT and PEG according to the formulation of Table 5. The procedure used was similar to Example 3A.

TABLE 5
The materials for the impregnation, Example 3D

| | Quantity in | |
|---|---|---|
| Name of Materials: | 5 mL dosage | |
| BHT | 0.2 | mg |
| PEG 3350 | 14 | mg |
| The drug-resin complex | 97 | mg |
| from Example 1B | | |

3E. Impregnating with BHT (0.4 mg) and PEG 3350

The alkaline treated dextromethorphan-polystirex complex from Example 1B was impregnated with BHT and PEG according to the formulation of Table 6. The procedure used was similar to Example 3A.

TABLE 6
The materials for the impregnation, Example 3E

| | Quantity in | |
|---|---|---|
| Name of Materials: | 5 mL dosage | |
| BHT | 0.4 | mg |
| PEG 3350 | 14 | mg |
| The drug-resin complex | 97 | mg |
| from Example 1B | | |

### EXAMPLE 4

### Orange Flavored Suspension Formulations and Stability Comparison

The drug-complexes from Example 3A, 3B, or 3C were formulated into orange flavored suspensions. Stability study was performed to evaluate the impact of using propylene glycol and/or phosphoric acid in the formulation, and the impact of using different drug-resin complex from Example 3A, 3B or 3C to prepare the suspension.

4A. Formulating an Orange Flavored Dextromethorphan-Polistyrex Suspension.

The drug-complex from Example 3A, 3B, or 3C was introduced to other components of the formulation as shown in Table 7 to prepare an orange flavored suspension. In practice, the amount of each ingredient used will be proportionally scaled up or scaled down based on the amount of the final product to be made.

TABLE 7
Composition of a dextromethorphan-
polistyrex suspension - Orange flavor

| # | Ingredient | mg/5 mL | Percent |
|---|---|---|---|
| 1 | Granulated beads from | 202.3 | 4.05 |
| | Example 3A, 3B, or 3C | | |
| 2 | citric acid | 3.0 | 0.060 |
| 3 | FD&C Yellow No. 6, | 0.115 | 0.0023 |
| 4 | Orange flavor | 5.0 | 0.100 |
| 5 | High fructose corn syrup | 1100.0 | 22.000 |
| 6 | Methylparaben | 7.5 | 0.150 |
| 7 | Propylparaben | 1.5 | 0.030 |
| 8 | Polysorbate 80 | 2.5 | 0.050 |
| 9 | Sucrose | 225.0 | 4.500 |
| 10 | Tragacanth | 22.5 | 0.450 |
| 11 | Xanthan gum | 5.0 | 0.100 |
| 12 | Propylene glycol | 150.0 | 3.00 |
| 13 | 90% Phosphoric acid | 5.0 | 0.100 |
| 14 | Water (qs)(mL) | 3.25 | 64.993 |
| | Total | 5.0 mL | 100 |

The formulation procedure is as follows: in a first container, tragacanth and xanthan gum were added to deionized water while mixing. Continued mixing for 20 minutes. High fructose corn syrup was added to the above mixture and stirred for 5 minutes to make a bulk liquid. In a second container, sucrose was added to deionized water to make a sucrose solution, to which citric acid was added and mixed until fully dissolved. The sucrose and citric acid solution was then added to the bulk liquid in the first container and stirred for 5 minutes, followed by adding methylparaben and propylparaben and mixed for 5 minutes to form a final bulk liquid. In a third container, FD&C Yellow was dissolved in deionized water and mixed for 5 minutes, to which were added orange flavor, polysorbate 80, an aqueous solution of phosphoric acid and propylene glycol, and the granulated dextromethorphan-resin particles (Example 3A, 3B, or 3C), and well mixed to form a polysorbate/resin mixture. The polysorbate/resin mixture was then added to the final bulk solution in the first container and mixed slowly for 5 minutes to yield the orange flavored suspension.

4B. Formulating an Orange Flavored Dextromethorphan-Polistyrex Suspension Without Propylene Glycol in the Formulation

The drug-complex from Example 3A, 3B, or 3C was granulated and introduced to other components of the formulation as shown in Table 7, however, without propylene glycol. The procedure for manufacturing is similar to Example 4A.

4C. Formulating an Orange Flavored Dextromethorphan-Polistyrex Suspension Without Propylene Glycol and Phosphoric Acid in the Formulation

The drug-complex from Example 3A, 3B, or 3C was granulated and introduced to other components of the formulation as shown in Table 7, however, without propylene glycol and phosphoric acid. The procedure for manufacturing is similar to Example 4A.

4D. Formulating an Orange Flavored Dextromethorphan-Polistyrex Suspension Without Propylene Glycol

The drug-complex from Example 3A, 3B, or 3C was granulated and introduced to other components of the formulation as shown in Table 7, however, without phosphoric acid. The procedure for manufacturing is similar to Example 4A.

Stability Comparison:

The stability of the suspensions, with and without propylene glycol and/or phosphoric acid, in the formulation, is presented in Table 8. Table 8 also illustrates the impact of using different drug-resin complex from Example 3A, 3B or 3C on the stability. Stability testing at 2 weeks and 4 weeks after storing at 50° C. and one month after storing at 40° C. were performed to establish stability. The stability test measures the % of the degraded product, the dextromethorphan ketone, as identified by HPLC.

TABLE 8
Total degradation (%) of the dextromethorphan-
polistyrex suspension - Orange flavor

| Sample | 2W_50° C. | 4W_50° C. | 1M-40° C. |
|---|---|---|---|
| Delsym ® Orange flavor 8 Oz | 0.389 | 0.664 | 0.533 |

| | Key agents in | Resin | Suspension | | | |
|---|---|---|---|---|---|---|
| # | formulation* | formulation | formulation: | 2W_50° C. | 4W_50° C. | 1M-40° C. |
| 1 | L-Methinione, | Example 3A | Example 4A | 0.311 | 0.65 | 0.127 |
| | BHT, PA, PG | | | | | |
| 2 | L-Methinione, | Example 3A | Example 4B | 0.237 | 0.558 | 0.119 |
| | BHT, PA | | | | | |
| 3 | L-Methinione, | Example 3A | Example 4C | 0.383 | 0.812 | 0.173 |
| | BHT | | | | | |
| 4 | L-Methinione, | Example 3A | Example 4D | 0.853 | 1.444 | 0.312 |
| | BHT, PG | | | | | |
| 5 | L-Methinione, | Example 3B | Example 4C | 0.402 | 0.623 | 1.7 |
| 6 | L-Methinione, | Example 3B | Example 4D | 0.992 | 1.784 | 0.329 |
| | PG | | | | | |
| 7 | L-Methinione, | Example 3C | Example 4C | 0.472 | 0.591 | 0.179 |
| | BHT (0.4 mg) | | | | | |
| 8 | L-Methinione, | Example 3C | Example 4D | 1.006 | 1.658 | 0.317 |
| | BHT (0.4 mg), | | | | | |
| | PG | | | | | |
| 9 | L-Methinione, | Example 3A | Example 4A | 0.863 | 1.334 | 0.269 |
| | BHT, PA, PG | | | | | |
| | (repeat) | | | | | |
| 10 | L-Methinione, | Example 3A | Example 4B | 0.241 | 0.449 | 0.141 |
| | BHT, PA | | | | | |
| | (repeat) | | | | | |
| 11 | BHT, PA, PG | Example 3D | Example 4A | 2.371 | — | — |
| 12 | BHT, PA | Example 3D | Example 4B | 3.406 | — | — |
| 13 | BHT, PEG, PG | Example 3E | Example 4C | 12.95 | — | — |
| 14 | BHT, PEG | Example 3E | Example 4D | 1.30 | — | — |

*BHT is a short form for Butylated hydroxytoluene; PG is a short form for propylene glycol; and PA is a short form for phosphoric acid.

The data in Table 8 indicates that the absence of propylene glycol and the presence of phosphoric acid and L-Methinione in the formulation stabilize the formation. Additionally, a drug-resin complex containing BHT at 0.2mg level and L-methionine has a positive effect of stability on the final suspension. Compared to the commercial Delsym ® Orange flavor, the present invention (Entries #1, 2, 9 and 10) achieved improved stability.

### EXAMPLE 5

### Grape Flavored Suspension Formulations and Stability Comparison

The drug-complexes from Example 3A, 3B, or 3C were formulated into grape flavored suspensions. Stability study was performed to evaluate the impact of using propylene glycol and/or phosphoric acid in the formulation, and the impact of using different drug-resin complex from Example 3A, 3B or 3C to prepare the suspension.

5A. Formulating a Grape Flavored Dextromethorphan-Polistyrex Suspension.

The drug-complex from Example 3A, 3B, or 3C was introduced to other components of the formulation as shown in Table 9 to prepare a grape flavored suspension. It is within the preview of a skilled artisan that the amount of each ingredient used can be proportionally scaled up or scaled down based on the amount of the final product to be made. The manufacturing procedure is similar to Example 4A.

TABLE 9
Composition of a dextromethorphan-polistyrex
suspension - Grape flavor

| # | Ingredient | mg/5 mL | Percent |
|---|---|---|---|
| 1 | Granulated beads from | 202.3 | 4.05 |
| | Example 3A, 3B, or 3C | | |
| 2 | citric acid | 3.0 | 0.060 |
| 3 | FD&C Blue | 0.473 | 0.0095 |
| 4 | D&C Red | 0.372 | 0.0074 |
| 5 | Grape Flavor | 10.0 | 0.200 |
| 6 | High fructose corn syrup | 1100.0 | 22.000 |
| 7 | Methylparaben | 7.5 | 0.150 |
| 8 | Propylparaben | 1.5 | 0.030 |
| 9 | Polysorbate 80 | 2.5 | 0.050 |
| 10 | Sucrose | 225.0 | 4.500 |
| 11 | Tragacanth | 22.5 | 0.450 |
| 12 | Xanthan gum | 5.0 | 0.100 |
| 13 | 90% Phosphoric acid | 5.0 | 0.100 |
| 14 | Water (qs) (mL) | 3.422 | 68.434 |
| | Total | 5.0 mL | 100 |

5B. Formulating a Grape Flavored Dextromethorphan-Polistyrex Suspension Without Propylene Glycol in the Formulation

The drug-complex from Example 3A, 3B, or 3C was granulated and introduced to other components of the formulation as shown in Table 9, however, without propylene glycol. The procedure for manufacturing is similar to Example 4A.

5C. Formulating a Grape Flavored Dextromethorphan-Polistyrex Suspension Without Propylene Glycol and Phosphoric Acid in the Formulation

The drug-complex from Example 3A, 3B, or 3C was granulated and introduced to other components of the formulation as shown in Table 9, however, without propylene glycol and phosphoric acid. The procedure for manufacturing is similar to Example 4A.

5. Formulating a Grape Flavored Dextromethorphan-Polistyrex Suspension Without Propylene Glycol

The drug-complex from Example 3A, 3B, or 3C was granulated and introduced to other components of the formulation as shown in Table 9, however, without phosphoric acid. The procedure for manufacturing is similar to Example 4A.

Stability Comparison:

The stability of the suspensions, with and without propylene glycol and/or phosphoric acid, in the formulation, is presented in Table 10. Table 10 also illustrates the impact of using different drug-resin complex from Example 3A, 3B or 3C on the stability. Stability testing at 2 weeks and 4 weeks after storing at 50° C. and one month after storing at 40° C. were performed to establish stability. The stability test measures the % of the degraded product, the dextromethorphan ketone, as identified by HPLC.

TABLE 10
Total degradation (%) of the dextromethorphan-polistyrex suspension - Grape flavor

| | Key agents in | Bead | Suspension | | | |
|---|---|---|---|---|---|---|
| # | formulation* | formulation | formulation: | 2W_50° C. | 4W_50° C. | 1M-40° C. |
| 1 | L-Methinione, | Example 3A | Example 5A | 0.981 | 0.803 | 0.244 |
| | BHT, PA, PG | | | | | |
| 2 | L-Methinione, | Example 3A | Example 5B | 0.485 | 0.54 | 0.175 |
| | BHT, PA | | | | | |
| 3 | L-Methinione, | Example 3B | Example 5A | 0.957 | 0.897 | 0.291 |
| | PA, PG | | | | | |
| 4 | L-Methinione, | Example 3B | Example 5B | 0.555 | 0.578 | 0.184 |
| | PA | | | | | |
| 5 | L-Methinione | Example 3B | Example 5C | 0.748 | 0.62 | 0.23 |
| 6 | L-Methinione, | Example 3B | Example 5D | 1.426 | 1.112 | 0.272 |
| | PG | | | | | |

*BHT is a short form for Butylated hydroxytoluene; PG is a short form for propylene glycol; and PA is a short form for phosphoric acid.

The data in Table 10 indicates that the absence of propylene glycol and the presence of phosphoric acid in the formulation stabilize the formation. Additionally, a drug-resin complex containing BHT at 0.2 mg level and L-methionine has a positive effect of stability on the final suspension.

### EXAMPLE 6

### Preparation of A Dextromethorphan Extended Release Ion Exchange Complex and Dissolution Study

Preparation of an Extended Release Drug-Resin Complex

A dextromethorphan extended release ion exchange complex was prepared by coating the alkalized drug-resin complex from Example 1B with a seal coat followed by coating with a functional coat in accordance with the ingredients in Table 11. A person skilled in the art would understand that in practice the amount of each ingredient in Table 11 will be proportionally scaled up or scaled down based on the amount of the final dosage form to be made.

TABLE 11
Coating composition

| | | | |
|---|---|---|---|
| # | Ingredient | mg/5 mL | % |

Seal coat

| | | | |
|---|---|---|---|
| 1 | HPC | 10 | 4.9 |
| 2 | PEG3350 | 1.5 | 0.7 |

| | | |
|---|---|---|
| 3 | Water | q.s. to 10% Solids |

Functional Coating

| | | | |
|---|---|---|---|
| 4 | Ethocel 10 cps | 42 | 20.6 |
| 5 | Tri ethyl citrate | 6.3 | 3.1 |
| | IPA:MeCl2 (60:40) Total | (5% solids) | |
| 6 | DI Water | 2% | |
| 7 | IPA | 60% | |
| 8 | Methylene Chloride | 40% | |
| 9 | Coated system | 204.1 | 100.00 |

For the seal coating, a coating solution was made by adding HPC and PEG3350 in water and gently stirring until dissolved. For the functional coating, a coating solution was prepared by combining the ingredients in the table above. The suspension was filtered through a #100 mesh screen and kept under constant stirring during the coating procedure. Coating was carried out in a fluid bed coating apparatus equipped with a Wurster Column (Niro MP-1). The coating parameters are as follows: uncoated drug-resin complex, 600 g; atomizing air pressure, 1.0 bar; nozzle size, 1.0 mm; spray rate, 6-12 g/min; product temperature, 21-25° C. Samples were collected at three intervals in order to assess how the coating weight gain influenced release. Following coating, the product was well mixed with colloidal silicon dioxide at 1%. Finally, the coated particles were cured in a forced draft oven for 48 hours at 40° C.

Dissolution Study

Drug release profile of the dextromethorphan extended release ion exchange complex from Example 6 was determined at 37° C. by adding the drug-resin complex containing 30 mg equivalent of dextromethorphan HBr to 750 mL 0.1 N HCL in a dissolution vessel equipped with paddles rotating at 50 rpm. After 1 hour, the pH of the solution was changed to 6.8 in situ by the addition of 250 mL of 0.2 M tribasic sodium phosphate buffer. Samples were withdrawn periodically from the dissolution apparatus using an automated sampler and analyzed via HPLC.

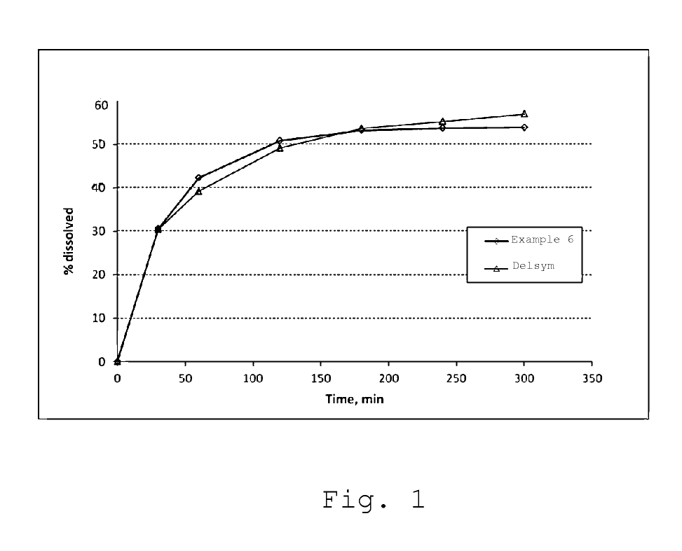

The dissolution profile from the study was illustrated in FIG. 1. FIG. 1 also illustrates the dissolution profile of the commercial Delsym. According to FIG. 1, about 50% the extended release complex of the present invention dissolved in about 5 hours. Importantly, this dissolution profile is substantially similar to that of the commercial Delsym.

The above description is provided for the purpose of describing embodiments of the invention and is not intended to limit the scope of the invention in any way. It will be apparent to those skilled in the art that various modifications and variations can be made in the drug-resin complexes stabilized by alkalizing agents, their methods of manufacture, and their uses without departing from the spirit or scope of the invention. Thus, it is intended that the present invention cover the modifications and variations of the invention provided they come within the scope of the appended claims and their equivalents.

What is claimed is:

1. A stabilized pharmaceutical composition comprising a drug-resin complex and L-methionine,
wherein the resin is treated with an inorganic alkaline material prior to forming the drug-resin complex.

2. The composition of claim 1, wherein the inorganic alkaline material is an alkali metal, an alkaline earth or post transition metal in form of oxide, hydroxide, carbonate or bicarbonate.

3. The composition of claim 2, wherein the inorganic alkaline material is sodium hydroxide.

4. The composition of claim 1, wherein the resin is a cationic exchange resin.

5. The composition of claim 4, wherein the resin is divinylbenzene sulfonic acid cationic resin.

6. The composition of claim 1, wherein the drug is prone to degradation.

7. The composition of claim 6, wherein the drug selected from a group consisting of dextromethorphan, codeine, morphine, hydrcodone, pseudoephedrine, phenylpropanolamine, and the salts thereof.

8. The composition of claim 7, wherein the drug is dextromethorphan.

9. The composition of claim 1 further comprising an antioxidant agent.

10. The composition of claim 9, wherein the antioxidant agent is butylated hydroxyanisole or butylated hydroxytoluene.

11. The composition of claim 10, wherein the antioxidant agent is butylated hydroxytoluene.

12. The composition of claim 9, further comprising phosphoric acid.

13. The composition of claim 12, wherein the composition does not include propylene glycol.

14. A stabilized pharmaceutical composition comprising a drug-resin complex,
wherein the resin is treated with an inorganic alkaline material prior to forming the drug-resin complex, and
wherein the drug-resin complex is coated with a diffusion barrier coat.

15. The composition of claim 14, wherein the diffusion barrier coat allows for sustained release of the drug.

16. The composition of claim 14, wherein the diffusion barrier coat allows for delayed release of the drug.

17. The composition of claim 14, wherein the inorganic alkaline material is an alkali metal, an alkaline earth or post transition metal in form of oxide, hydroxide, carbonate or bicarbonate.

18. The composition of claim 14, wherein the inorganic alkaline material is sodium hydroxide.

19. A stabilized pharmaceutical composition comprising a drug-resin complex,
wherein the resin is treated with an inorganic alkaline material prior to forming the drug-resin complex, and
wherein the drug-resin complex is impregnated with an alkalizing agent.

20. The composition of claim 19, wherein the inorganic alkaline material is an alkali metal, an alkaline earth or post transition metal in form of oxide, hydroxide, carbonate or bicarbonate.

21. The composition of claim 20, wherein the inorganic alkaline material is sodium hydroxide.

22. The composition of claim 19, wherein the alkalizing agent is selected from a group consisting of pseudoephedrine, phenylpropanolamine, MgO, Mg(OH)₂, CaCO₃, Ca(OH)₂, and MgCO₃.

23. The composition of claim 22, wherein the alkalizing agent is MgO.

24. The composition of claim 19, wherein the drug-resin complex is further impregnated with a solvating agent.

25. The composition of claim 24, wherein the solvating agent is selected from a group consisting of polyethylene glycol, propylene glycol, and glycerol.

26. A method for preparing a pharmaceutical composition comprising:
a) contacting a resin with an inorganic alkaline material in an aqueous medium to obtain a treated resin;
b) combining a drug and the treated resin in a liquid to form a drug-resin complex;
c) formulating the alkalized drug-resin complex to form a pharmaceutical composition.

27. The method of claim 26, wherein the inorganic alkaline material is an alkali metal, an alkaline earth or post transition metal in form of oxide, hydroxide, carbonate or bicarbonate.

28. The method of claim 26, wherein the inorganic alkaline material is sodium hydroxide.

29. The method of claim 26, further comprising the step of impregnating the drug-resin complex with magnesium oxide after step b) of forming the drug-resin complex and prior to step c) of the formulation.

30. The method of claim 26, further comprising the step of impregnating the drug-resin complex with L-methionine after step b) of forming the drug-resin complex and prior to step c) of the formulation.

31. The method of claim 26, further comprising the step of impregnating the drug-resin complex with L-methionine and butylated hydroxytoluene after step b) of forming the drug-resin complex and prior to step c) of the formulation.

32. The method of claim 31, wherein the formulating step comprising the adding of phosphoric acid.

33. The method of claim 26, further comprising the step of coating the drug-resin complex with a diffusion barrier coat after step b) of forming the drug-resin complex and prior to step c) of the formulation.
